(19) United States
(12) Patent Application Publication
(10) Pub. No.: US 2023/0115636 A1
(43) Pub. Date: Apr. 13, 2023

(54) CATHETER BAG MANAGEMENT SYSTEM

(71) Applicant: Michael E. Coughlin, Mission Hills, KS (US)

(72) Inventor: Michael E. Coughlin, Mission Hills, KS (US)

(21) Appl. No.: 17/518,736

(22) Filed: Nov. 4, 2021

Related U.S. Application Data
(60) Provisional application No. 63/254,594, filed on Oct. 12, 2021.

(51) Int. Cl. A61F 5/44

(52) U.S. Cl. CPC A61F 5/4408; A61M 25/0017

(57) ABSTRACT

A support system for a urine collection bag includes a waistband; a strap having a first end connected to the urine collection bag and a second end connected to the belt; and a fastener on the second end of the strap. The waistband and strap support the weight of the urine collection bag so it doesn't creep down the patient's leg and tug on the catheter and permits the patient to reposition the urine collection bag quickly and easily.

### RELATED APPLICATION

The present application is a non-provisional patent application and claims priority benefit, with regard to all common subject matter, of earlier-filed U.S. provisional patent application titled “FOLEY CATHETER BAG MANAGEMENT SYSTEM”, Ser. No. 63/254,594, filed on Oct. 12, 2021, incorporated by reference in its entirety into the present application.

### BACKGROUND

A Foley catheter is a thin, sterile tube that is inserted in a patient's bladder to drain urine into a urine collection bag. A balloon on the end of the catheter is inflated with air or sterile water after insertion to serve as an anchor to hold the catheter in place. These and similar catheters are often used for urinary hesitancy problems, urethra obstructions, urine output monitoring, sterile urine collection procedures, nerve-related bladder dysfunctions, prostate problems, and other medical uses.

As the urine collection bag fills with urine, it becomes heavy and tends to tug on the catheter. This can be painful and can irritate the patient's bladder and urinary tract. If the urine collection bag is dropped or moved significantly, it can completely dislodge the catheter from the patient's bladder, causing extreme pain and serious damage to the bladder and urinary tract. To prevent this, the urine collection bag is typically attached to one of patient's legs with tape and/or elastic straps. Unfortunately, with this attachment method, the urine collection bag often creeps down the patient's leg as the patient moves, requiring the patient to bend over and hold the bag while attempting to re-apply the tape or re-adjust the straps. Sometimes, the bag creeps so far that the inflated balloon in the patient's bladder serves as a brake, causing the aforementioned problems. The tape or straps can also irritate the patient's skin, especially when frequently attached and reattached, and can inhibit blood circulation when applied too tightly. Taping or strapping the bag to the patient's leg also pulls the tube toward the leg, causing discomfort for some patients, especially males. Tape also makes bathing more cumbersome.

Patients also often wish to reposition a urine collection bag to a more comfortable position depending on whether they are standing, walking, sitting, or lying down. But attaching the bag with tape and/or straps makes it difficult and time consuming to do so because the tape or straps must be removed and reapplied to reposition the bag.

### SUMMARY

The present invention solves the above-described problems and related problems by providing an improved support system for a urine collection bag used with Foley catheters and other fluid catheters. The support system more securely supports the urine collection bag, prevents the bag from tugging on its catheter, and permits the bag to be quickly and easily repositioned for maximum comfort.

An embodiment of the support system comprises a belt configured to be worn around a patient's waist; a strap having a first end connected to a urine collection bag and a second end connected to the belt; and a fastener such as Velcro on the second end of the strap. In one embodiment, the second end of strap is doubled over in a loop that encircles the belt.

Because the belt is securely wrapped around the patient's waist and the strap suspends from and is supported by the belt, the belt and strap support the weight of the urine collection bag and prevent it from creeping down the patient's leg and tugging on the catheter. This eliminates the need to attach the bag to the patient's leg with tape or straps, although some patients may still desire to use some tape or straps to prevent swaying of the bag when wearing loose clothes.

The support system also permits the patient to reposition the urine collection bag quickly and easily both horizontally/laterally and vertically to an endless number of positions. To reposition the bag laterally, the strap may be moved left or right on the belt by moving the second end of the strap on the belt. This allows the bag to be suspended adjacent different portions of the patient's leg or to even be moved to the patient's opposite leg.

To raise the bag on the patient's leg, the effective length of the strap is shortened by unfastening the fastener, pulling the tail end of the strap down closer to the bag, and refastening the fastener. Conversely, to lower the bag, the effective length of the strap is increased by unfastening the fastener, lifting the tail end of the strap up closer to the belt, and refastening the fastener. The bag may also be repositioned vertically by simply moving the belt up or down on the patient's torso.

A patient may make the above-described adjustments without removing his or her pants or other leg coverings, because the strap can be easily accessed from under the patient's shirt or blouse. Moreover, while these adjustments are being made, the weight of the urine collection bag is still supported by the waistband and the strap so the bag doesn't tug on the catheter. And because the bag is supported without tape, the patient can more easily bathe.

This summary is provided to introduce a selection of concepts in a simplified form that are further described in the detailed description below. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 is an elevational view depicting a prior art method for securing a urine collection bag to a patient.

FIG. 2 is an elevational view of a urine collection bag support system constructed in accordance with embodiments of the invention and shown supporting a urine collection bag in a first position.

FIG. 3 is an elevational view of the urine collection bag support system shown supporting a urine collection bag in a second position.

FIG. 4 is an elevational view of the urine collection bag support system shown supporting a urine collection bag in a third position.

FIG. 5 is an elevational view of the urine collection bag support system shown supporting a urine collection bag in a fourth position.

FIG. 6 is an elevational view of the urine collection bag support system shown supporting a urine collection bag in a fifth position.

The drawing figures do not limit the present invention to the specific embodiments disclosed and described herein. The drawings are not necessarily to scale, emphasis instead being placed upon clearly illustrating the principles of the invention.

### DETAILED DESCRIPTION

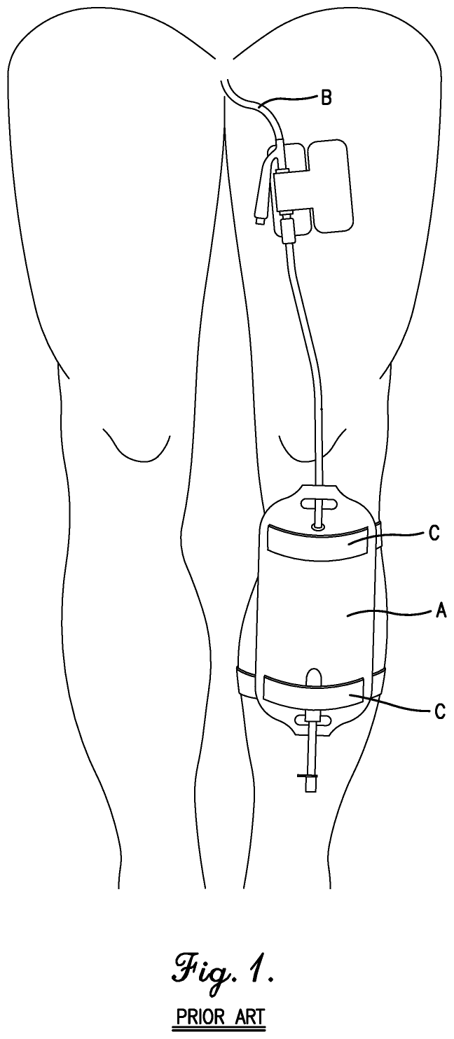

FIG. 1 depicts a prior art method for securing a urine collection bag A to a patient in which a catheter B has been inserted. As shown, the bag A is attached to one of the patient's legs with tape and/or elastic straps C. With this attachment method, the urine collection bag A often creeps down the patient's leg as the patient moves and/or as the bag fills with urine, requiring the patient to bend over and hold the bag while attempting to re-apply the tape or re-adjust the straps. Sometimes the bag creeps so far that it tugs on the catheter, with the inflated balloon in the patient's bladder serving as a brake, causing much discomfort and pain and occasionally dislodging the catheter from the bladder. This attachment method also makes is difficult and time consuming to reposition the bag, because the tape and/or straps must be removed and reapplied while the patient holds the urine collection bag. All of these steps are complicated when the patient is wearing clothes because the patient must partially undress to handle the urine collection bag, the tape and/or straps, and the associated catheter.

The present invention provides an improved support system for a urine collection bag that more securely supports the bag while permitting it to be quickly and easily repositioned for maximum comfort.

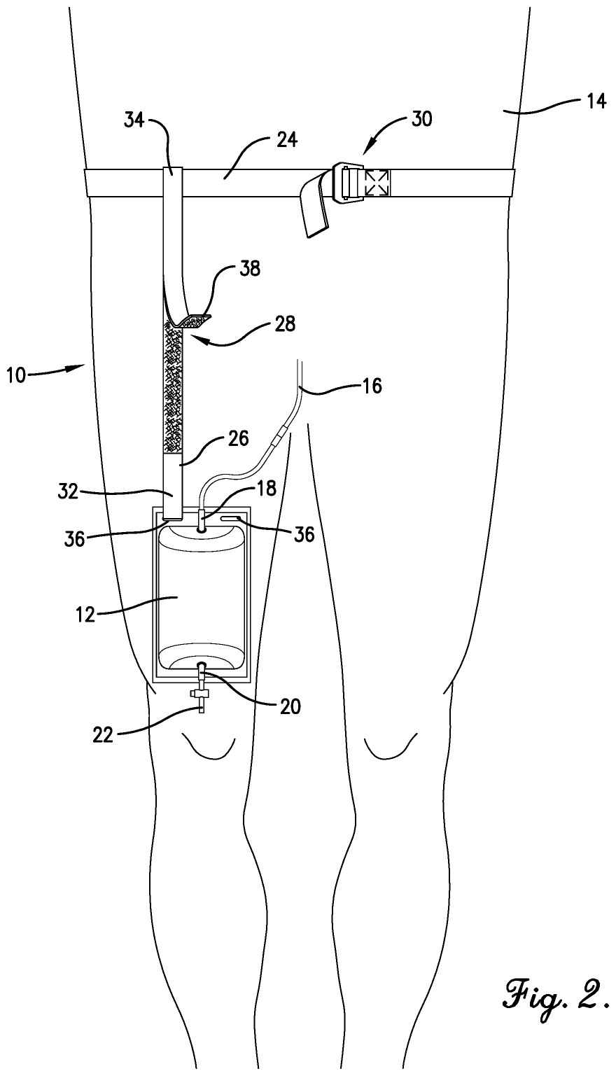

Referring initially to FIG. 2, an embodiment of the support system 10 is shown supporting a urine collection bag 12 to a patient 14. A catheter 16 is attached to the bag at one end and inserted in the patient's bladder at the opposite end. The urine collection bag may be any conventional medical bag and may include a fill port 18 for coupling with the catheter and a drain port 20 for coupling with a drain tube 22 or container. The catheter 16 may be a Foley catheter or any other fluid-carrying tube. As used herein, the term “patient” encompasses any user of the urine collection bag, male or female.

An embodiment of the support system 10 broadly comprises a belt 24, a strap 26, and a fastener 28. The belt 24 is preferably a waistband configured to be worn around a patient's waist, but it may also be in the form of suspenders, a vest, or any other item worn on or near the patient's torso and to which the strap may be attached. In one embodiment, the belt is formed of cloth or elastic and has a buckle 30 or other mechanism that allows it to be tightened or loosened.

The strap 26 is preferably formed of cloth or other strong but flexible materials and has a first end 32 directly or indirectly connected to the urine collection bag 12 and a second end 34 that is directly or indirectly connected to the belt. In one embodiment, the first end 32 is sewn on or otherwise semi-permanently attached to the urine collection bag. In other embodiments, the first end 32 may be removably attached to the urine collection bag with Velcro or other fasteners. As shown in FIG. 2, the top of the bag 12 may have one or more slots 36 through which the first end 32 of the strap may be inserted. In some embodiments, the strap may be moved between the slots.

The second end 34 of the strap may be attached to the belt 24 in a variety of ways. In one preferred embodiment, the second end of the strap is doubled over the remainder of the strap to form a loop that encircles the belt as shown in FIG. 2.

The fastener 28 is attached to the second end 34 of the strap to secure the strap 26 on the belt 24 and to permit adjustment of the strap relative to the belt. In one embodiment, the fastener comprises Velcro or other interlocking hook and loop materials on one side of the belt that may be pressed together to hold the strap on the belt and pulled apart, repositioned, and reattached to change the effective length of the strap. Other embodiments of the fastener 28 may include buttons, snaps, zippers, or any other mechanisms that can attach the strap to the belt while permitting adjustment of the position of the strap on the belt and the effective length of the strap.

Use of the support system 10 will now be described. First, the belt 24 is wrapped around the patient's waist and tightened or loosened as-needed. Then, the first end 32 of the strap is connected to the urine collection bag (unless the strap is already sewn on the bag), and the second end of the strap 34 is wrapped around and attached to the belt. The order of these steps is not critical and can be changed without departing from the scope of the invention. At this point, the belt and strap support the weight of the bag whenever the patient stands or is upright so the bag doesn't creep down the patient's leg and tug on the catheter 16. The bag may also be taped to the patient's leg to prevent it from moving too much, but pressure exerted by the patient's pants or other leg coverings are typically sufficient to resist movement of the bag.

The support system 10 may be adjusted in several ways to reposition the bag 12 both laterally/horizontally and vertically with respect to the patient. Applicant has discovered that certain positions of the bag are more comfortable when a patient is performing certain activities or is in particular positions. For example, when the patient is laying in a supine position while sleeping or resting, the patient may be more comfortable if the catheter tube 16 is stretched out and the bag 12 is lowered toward the patient's feet. When sitting in a chair, a patient may wish to raise the bag. When walking, a patient may be most comfortable when the bag is positioned to leave lots of slack in the catheter tube so as to exert no forces on the catheter.

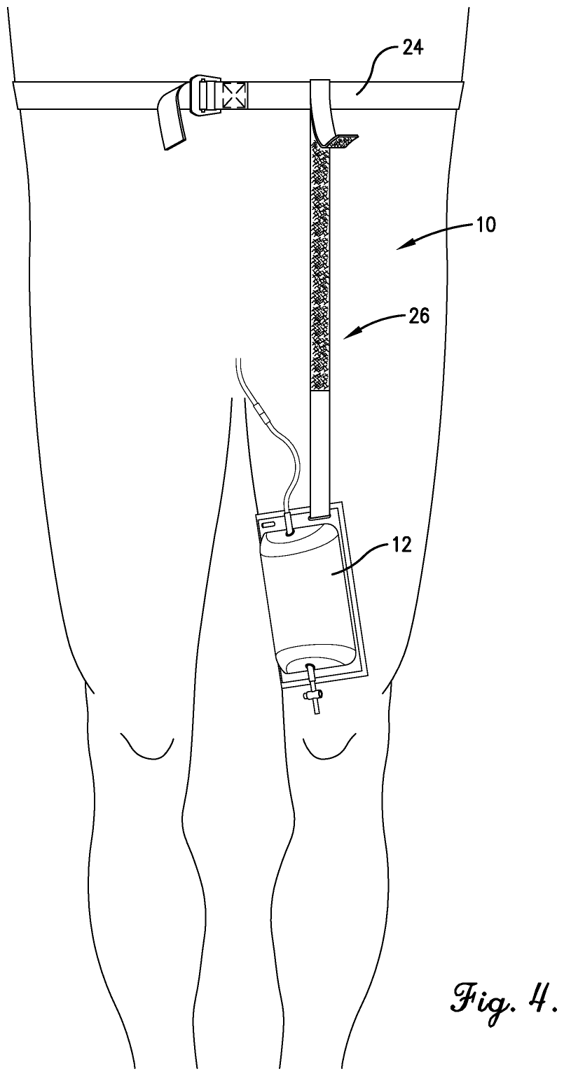

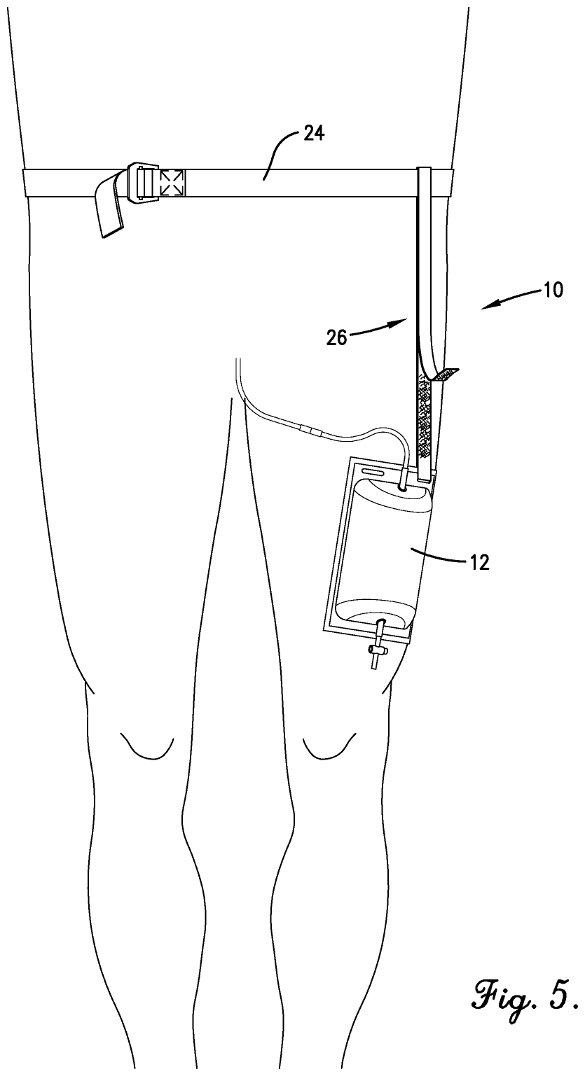

To reposition the bag laterally, the second end 34 of the strap may be moved left or right on the belt 24 by sliding it on the belt. This allows the bag to be suspended adjacent different portions of the patient's leg or to even be moved to the patient's opposite leg. FIGS. 2, 4, and 5 show several exemplary lateral positions of the bag. The bag may also be moved laterally by moving the first end of the strap between the slots 36 in the bag as shown in FIG. 2 vs. FIG. 5.

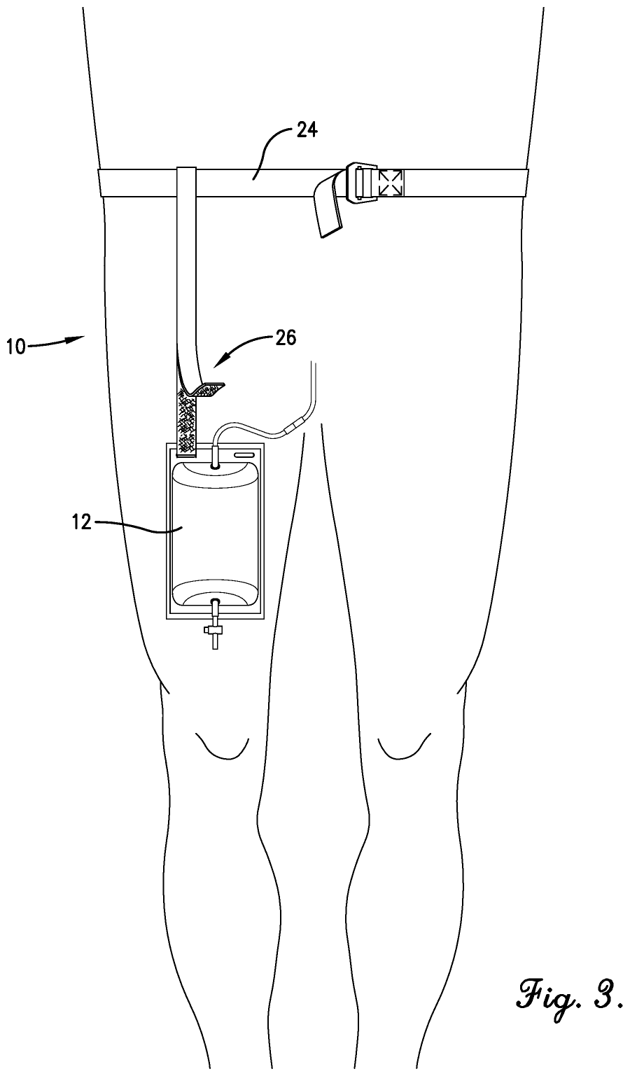

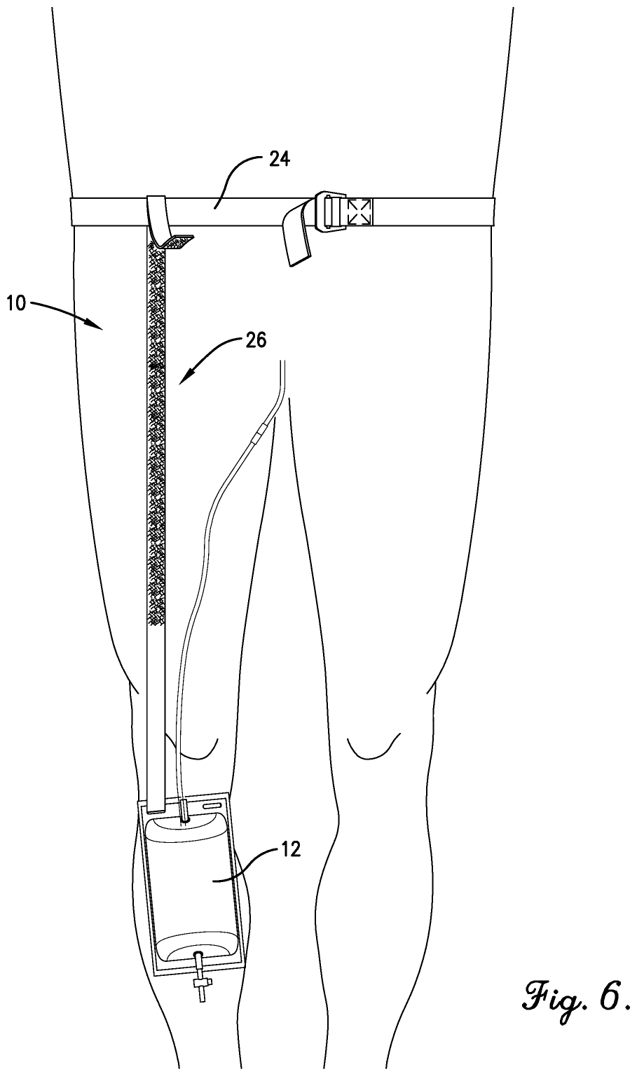

To raise the bag on the patient's leg, the doubled over portion of the strap may be separated, and a tail end 38 of the strap 26 may be pulled down so it is closer to the bag 12 to shorten the effective length of the strap. The doubled over portion of the strap may then be refastened. Conversely, to lower the bag on the patient's leg, the doubled over portion of the strap is separated, and the tail end 38 of the strap is moved up so it is closer to belt to lengthen the strap. The doubled over portion of the strap may then be refastened. The bag may also be repositioned vertically by simply moving the belt up or down on the patient's torso. FIGS. 2-6 show several exemplary vertical positions of the bag.

Although the drawing figures illustrate a few different positions to which the bag may be moved, the support system 10 allows the bag to be supported in an endless number of different positions relative to the patient.

Advantageously, a patient may make the above-described adjustments without removing his or her pants or other leg coverings because the strap and belt can be accessed and manipulated by lifting the patient's shirt or other torso-covering garment. Moreover, while the above-described adjustments are being made, the belt and strap continue to support the weight of the urine collection bag so it doesn't tug on the catheter.

### Additional Considerations

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the current technology can include a variety of combinations and/or integrations of the embodiments described herein.

Although the present application sets forth a detailed description of numerous different embodiments, the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A support system for supporting a urine collection bag on a patient, the support system comprising:
a belt configured to be worn by the patient;
a strap having a first end directly or indirectly connected to the urine collection bag and a second end directly or indirectly connected to the belt; and
a fastener coupled with the strap that permits the belt and the strap to support the urine collection bag in a plurality of different lateral and vertical positions relative to the patient.

2. The support system of claim 1, wherein the belt is a waistband configured to be worn around a waist of the patient.

3. The support system of claim 2, wherein the waistband is formed of cloth and includes a buckle to tighten or loosen the waistband on the patient's waist.

4. The support system of claim 2, wherein the waistband is configured to directly contact the patient's waist and can be shifted vertically up and down with respect to the patient's waist.

5. The support system of claim 1, wherein the strap is formed of cloth.

6. The support system of claim 1, wherein the second end of the strap encircles the belt.

7. The support system of claim 1, wherein the fastener includes hook and loop fastening elements on the strap that permit an effective length of the strap to be adjusted.

8. A support system for supporting a urine collection bag on a patient, the support system comprising:
a waistband configured to be worn around a waist of the patient;
a strap having a first end connected to the urine collection bag and a second end that encircles the belt; and
hook and look fasteners on the second end of the strap that may be selectively unfastened and refastened to permit the strap to be shifted horizontally with respect to the belt and to permit the first end of the strap to be raised or lowered with respect to the belt.

9. The support system of claim 8, wherein the waistband is formed of cloth and includes a buckle to tighten or loosen the waistband on the patient's waist.

10. The support system of claim 8, wherein the waistband is configured to directly contact the patient's waist and can be shifted vertically up and down with respect to the patient's waist.

11. The support system of claim 8, wherein the strap is formed of cloth.

12. The support system of claim 8, wherein the second end of the strap wraps around the belt to encircle the belt.

13. A catheter bag system for collecting urine from a bladder of a patient, the system comprising:
a urine collection bag;
a catheter having a first end configured to be inserted in the bladder of the patient and a second end configured to be attached to the urine collection bag to direct urine from the bladder to the urine collection bag;
a support system for supporting the urine collection bag to the patient, the support system comprising:
a belt configured to be worn by the patient;
a strap having a first end directly or indirectly connected to the urine collection bag and a second end directly or indirectly connected to the belt;
a fastener coupled with the strap that permits the strap to be shifted horizontally with respect to the belt and that permits the first end of the strap to be shifted vertically with respect to the belt.

14. The catheter bag system of claim 13, wherein the belt is a waistband configured to be worn around a waist of the patient.

15. The catheter bag system of claim 14, wherein the waistband is formed of cloth and includes a buckle to tighten or loosen the waistband on the patient's waist.

16. The catheter bag system of claim 14, wherein the waistband is configured to directly contact the patient's waist and can be shifted vertically up and down with respect to the patient's waist.

17. The catheter bag system of claim 13, wherein the strap is formed of cloth.

18. The catheter bag system of claim 13, wherein the second end of the strap encircles the belt.

19. The catheter bag system of claim 13, wherein the fastener includes hook and loop fastening elements on the strap that permit an effective length of the strap to be adjusted.

20. The catheter bag system of claim 13, wherein the catheter is a Foley catheter.
